(19) United States
(12) Patent Application Publication
(10) Pub. No.: US 2019/0059733 A1
(43) Pub. Date: Feb. 28, 2019

(54) TREMOR REDUCTION DEVICE

(71) Applicants: 5IVE MICRONS, Fresno, CA (US); CALIFORNIA STATE UNIVERSITY, FRESNO FOUNDATION, Fresno, CA (US)

(72) Inventor: The Nguyen, Fresno, CA (US)

(73) Assignees: 5IVE MICRONS, Fresno, CA (US); CALIFORNIA STATE UNIVERSITY, FRESNO FOUNDATION, Fresno, CA (US)

(21) Appl. No.: 16/172,829

(22) Filed: Oct. 28, 2018

Related U.S. Application Data
(63) Continuation-in-part of application No. 15/601,201, filed on May 22, 2017.
(60) Provisional application No. 62/728,368, filed on Sep. 7, 2018.
(60) Provisional application No. 62/383,287, filed on Sep. 2, 2016.

(51) Int. Cl. A61B 5/00; A61B 5/11

(52) U.S. Cl. CPC A61B 5/0053; A61B 5/1101; A63B 21/22; A61B 5/4082; A61B 5/6824

(57) ABSTRACT

A wearable tremor reduction device reduces tremor by internally generating forces which cancel or reduce the magnitude force of the tremor experienced by the person wearing the device. The device may be worn on a wrist, arm, ankle or leg. The device has a plurality of housing members which are connected together. Each housing member contains a mass which is translatable along an axis between a proximal limit and a distal limit, and a neutral position approximately midway between the proximal limit and the distal limit. Following imposition of a force having a component along the axis, a biasing means returns the mass to the neutral position.

This application is a continuation-in-part of and claims priority to U.S. patent application Ser. No. 15/601,201, filed on May 22, 2017, which claims domestic priority to U.S. Provisional Application 62/383,287, filed Sep. 2, 2016. This application also claims domestic priority to U.S. Provisional Application 62/728,368, filed Sep. 7, 2018. U.S. patent application Ser. No. 15/601,201, U.S. Provisional Application 62/383,287, and U.S. Provisional Application 62/728,368 are incorporated herein in their entireties by this reference.

### FIELD OF THE INVENTION

The present invention relates to improved devices for dampening tremors or uncontrolled movements of the body, and specifically to dampening tremors of the hand.

### DISCUSSION OF THE BACKGROUND

Tremors of various extremities of the human body are common movement disorders, characterized by rhythmic oscillations of the extremity around one or more joints. One of the most important characteristics to be assessed in a patient's tremor is the tremor frequency, which is measured in oscillations per second or cycles per second (Hz). Another important characteristic of tremor is amplitude, which is the linear or angular displacement of the limb or body part. Tremor amplitude is measured in millimeters or degrees. Tremor frequency and amplitude can be measured to a relatively high degree of accuracy by known apparatus and methods.

Tremors which are caused by Parkinson's disease, i.e., Parkinsonian tremor, and essential tremor can significantly impact the quality of life of the person suffering from these maladies. Daily functions, such as eating, combing one's hair, brushing teeth, etc., which functions are generally taken for granted by persons without tremors—can be demanding and frustrating exercises for those suffering from tremors. Tremor magnitude is frequently reported as being the greater problem from persons suffering from the disorder, because it has a greater impact on a person's ability to perform these daily activities. Because of these issues, a variety of solutions to tremors have been proposed to provide relief from persons suffering from tremor.

While drug therapy has been employed to provide relief for persons suffering from these tremors, the expense and potential side effects of the treatment can be an obstacle. There have also been efforts to provide relief without using drugs utilizing electro-mechanical or mechanical devices. For example, one device utilizes a wearable tremor suppression exoskeleton, which appears as a robotic external structure. In the absence of external forces, this device applies dynamic internal forces on the upper limb programmed to reduce the tremor by applying biomechanical loads. Another device is a wearable orthosis which uses a DC motor to reduce tremors at each joint. Some tremor suppression devices attempt to suppress the tremors by application of pressure at a specific location on a human extremity to induce a tremor suppressing stimulus. Another device uses a forearm and wrist splint to attach at least one gyroscope. Other devices utilize braces having compartments containing a viscous fluid to dampen wrist flexion and extension tremor. Another device is a weighted glove which is customizable by size. Patients are able to adjust the weight of the glove by filling integrated pockets in the glove with circular weight disks.

With many of the known devices, the persons wearing the devices have complained that the devices are bulky, uncomfortable, too heavy, or simply did not provide a satisfactory solution to their tremor problem.

Thus, there is a need for a simple, relatively light, and effective device which suppresses tremors caused by Parkinson's disease and essential tremor.

### SUMMARY OF THE INVENTION

The present invention relates to a tremor reduction device and methods of using the same. Embodiments of the present invention provide a solution to the need identified above. Embodiments of the invention create neutralizing forces which cancel or reduce the magnitude of the tremor experienced by the person wearing the device. Parkinsonian tremor typically has a frequency in the range of 3-7 Hz, while essential tremor typically has a frequency in the range of 4-12 Hz. The tremors may have a vertical component, a horizontal component, and a rotational component.

In some embodiments, the tremor reduction device may comprise a plurality of housing members connected together and connected to the hand, wrist, and/or forearm of the user. The housing members may be constructed from at least one of a rigid polymer or plastic, a metal, a metal alloy, carbon fiber, composite materials, and the like. Each housing member may contain a mass which is translatable along an axis between a proximal limit and a distal limit, and a neutral position midway between the proximal limit and the distal limit, where, following imposition of a force having a component along the axis, a biasing means (e.g., a spring) operable to return the mass to the neutral position. The housing members and the translatable masses therein may be arranged to absorb vibrational energy generated by the user's tremors, such Parkinsonian or essential tremors. The housing members and translatable weights may be arranged around the hand, wrist, and/or forearm of the user such that the weights are translatable along different axes, such as along one or more of the x, y, and z axes so that the vibrational energy directed in different axial directions. For example, the tremor reduction device may include at least two housings having weights that are translatable along orthogonal axes. The tremor reduction device may include three housings each having weights that are translatable along substantially orthogonal axes, such that each weight moves along one axis in an about x, y, z arrangement of the axes. The tremor reduction device may further include additional housing members having axes for a translatable weight that are arranged at oblique angles to translatable weight axes of other housings (e.g., to axes of the translatable weights in the first, second, and/or third housing members) such that further vibrational directions are directly addressed by the device. In other embodiments, two or more housings weights may be arranged such that the weights therein are translatable along substantially parallel axes. In still further embodiments, the housings and translatable weights may be arranged such that some of the translatable weights move along substantially parallel axes, while others move along substantially orthogonal axes and/or substantially oblique axes.

In some embodiments, the plurality of housing members may comprise one or more housing members in close mechanical connection to the hand, wrist, and/or forearm of the user. The tremor reduction device may include at least two housing members (first and second housings) arranged around, e.g., the wearer's wrist. The first housing member may have a first proximal sidewall and a first distal sidewall, where the first proximal sidewall and the first distal sidewall are in opposite facing, spaced-apart relation. A first mass may be disposed between the first proximal side wall and the first distal side wall, wherein the first mass may have a first proximal side and a first distal side. A first proximal spring may be disposed between the first proximal side wall and the first proximal side of the first mass, and a first distal spring may be disposed between the first distal side wall and the first distal side of the first mass.

The second housing member may have a second proximal sidewall and a second distal sidewall in opposite facing, spaced-apart relation. A second mass may be disposed between the second proximal side wall and the second distal side wall, wherein the second mass has a second proximal side and a second distal side. A second proximal spring may be disposed between the second proximal side wall and the second proximal side of the second mass, and a second distal spring may be disposed between the second distal side wall and the second distal side of the second mass. The translatable weights of the first and second housings may be positioned substantially orthogonally relative to one another to absorb vibrational energies in different directions. In other embodiments, the translatable weight of the first housing may move along an axis that is substantially parallel to the axis of the translatable weight of the second housing.

The tremor reduction device may further include a third housing member having similar construction to the first and second housing members. The third housing member may have a third proximal sidewall and a third distal sidewall in opposite facing, spaced-apart relation. A third mass may be disposed between the third proximal side wall and the third distal side wall, wherein the third mass has a third proximal side and a third distal side. A third proximal spring may be disposed between the third proximal side wall and the third proximal side of the third mass, and a third distal spring may be disposed between the third distal side wall and the third distal side of the third mass. The third housing member may have a translatable weight that moves along an axis that is substantially orthogonal to both the axes of translatable weights in the first and second housings, which may be positioned substantially orthogonally relative to one another. In other embodiments, the translatable weight of the third housing member may move along an axis that is substantially parallel to one or both of the axes of the translatable weights of the first and second housings. In still other embodiments, the translatable weight of the third housing may move along an axis that is oblique to one or both of the axes of the translatable weights of the first and second housings.

The tremor reduction device may further include a fourth housing member having similar constructions to the first, second, and third housing members. The fourth housing member may have a fourth proximal sidewall and a fourth distal sidewall in opposite facing, spaced-apart relation. A fourth mass may be disposed between the fourth proximal side wall and the fourth distal side wall, wherein the fourth mass has a fourth proximal side and a fourth distal side. A fourth proximal spring may be disposed between the fourth proximal side wall and the fourth proximal side of the fourth mass, and a fourth distal spring may be disposed between the fourth distal side wall and the fourth distal side of the fourth mass. The fourth housing member may have a translatable weight that moves along an axis that is substantially orthogonal to at least one of the axes of the translatable weights in the first, second, and third housings. In other embodiments, the translatable weight of the third housing may move along an axis that is substantially parallel to one of the axes of the translatable weights of the first, second, and third housings. In still other embodiments, the translatable weight of the fourth housing may move along an axis that is oblique to one of the axes of the translatable weights of the first, second, and third housings. Further embodiments may include additional housing containing translatable weights that are positioned substantially parallel, perpendicular, or oblique to the other housings of the tremor reduction device.

In some embodiments, the plurality of housings may be connected to the wrist of the individual. For example, each of the four housings described above may be positioned on one side of the wrist (e.g., the first housing may be positioned on the lateral side of the wrist, the second housing may be positioned on the posterior side of the wrist, the third housing may be positioned on the medial side of the wrist, and the fourth housing may be positioned on the anterior side of the wrist). The housings may be held together around the wrist by fastening or binding structures between the housings, such as flexible straps, adjustable connectors, pivoting joints between the housings, combinations thereof, and/or other mechanisms.

In some embodiments, the first housing member may be flexibly linked to the second housing member, the second housing member may be flexibly linked to the third housing member, the third housing member may be flexibly linked to the fourth housing member, and the fourth housing member may be flexibly linked to the first housing member, thereby forming an interconnected band adapted to encircle a limb (i.e., a wrist) of the user.

In other embodiments, the present invention may provide a tremor reduction device comprising a frame structure that attaches to the hand, wrist, and/or forearm of the user and supports one or more housing members at a position adjacent to (e.g., lateral, medial, or distal to) the user's hand wrist, or forearm. The securing frame may comprise a coupling plate having a shape that is complementary to the portion of the user's body (e.g., a portion of the hand, a portion of the wrist, and/or a portion of the forearm), having contouring to accommodate the shape of the portion of the user's body, creating an ergonomic fit. For example, the coupling plate may comprise a shape which is complementary to the shape of a back side of a user's hand for an ergonomic fit (e.g., a substantially flat plate with gently curving downturned edges, the edges curving around the sides of the hand).

The coupling plate may further comprise an attachment element operable to secure the plate to the user's hand, wrist, and/or forearm. For example, the coupling plate may include securing straps that pass through a plurality of slots in the coupling plate and operable to cinched over the user's hand, wrist, and/or forearm to hold the securing frame in place on the user. The straps may include a fastening element such as a snap, a buckle, a hook, a hook and loop device (i.e., Velcro), and the like operable to releasably hold the straps in place around the hand, wrist, or forearm of a user. In some embodiments, the straps may comprise an elastic material operable to stretch around the hand, wrist, and/or forearm of the user and retract to hold the frame structure in place on the user.

The frame structure may include a scaffolding for connecting one or more brackets to the coupling plate. The scaffolding may extend from an edge or a surface of the coupling plate. For example, the scaffolding may extend medially, laterally or distally from the coupling plate, such that it holds the housing member(s) at a short distance from the coupling plate and the user's hand, wrist, or forearm. The scaffolding may include one or more brackets or attachment points, each operable to receive and secure a housing member containing a translatable weight. In some embodiments, the brackets or attachment points may each be engaged with one or more housing members secured thereto by fasteners. The one or more housings may be secured to the bracket or attachment point by at least one of a screw, a bolt, a rivet, pressure fitting, tab and slot fasteners, an adhesive, and/or other fastening mechanisms.

In some embodiments, the arrangement of the one or more brackets or attachment points on the scaffolding may position the housings such that the axes of the translatable weights within each housing are substantially parallel, substantially perpendicular, or oblique. For example, the scaffolding may include a first bracket that is lateral to the hand, wrist, or forearm, and a second bracket that is medial to the hand, wrist, or forearm, such that the housing members attached to the brackets and the axes of the translatable weights therein are on substantially parallel planes. The axes of the translatable weights in the first and second housing members may be substantially parallel or substantially perpendicular to each other. The scaffolding may include further brackets for positioning additional housing members such that the axis along which the translatable weight therein moves is substantially parallel, substantially perpendicular, or oblique to the axes of the translatable weights in the first and second housing members. For example, the scaffolding may include a third bracket that is posterior to the hand, wrist, or forearm of the user and perpendicular to the first and second brackets. A third housing member may be secured to the third bracket, positioning the third housing member such that an axis along which the translatable weight therein moves is perpendicular to the axes of the translatable weights in both of the first and second housings. In some embodiments, the scaffolding may include further brackets or attachment points to which additional housing members are attached having translatable weights that move along axes that are substantially parallel, substantially perpendicular, or oblique to the axes of the translatable weights in the other housing members attached to the scaffolding.

In a particular embodiment, the tremor reducing device may include two translatable masses that are arranged such that one translatable mass is lateral to the hand, wrist, or arm, and one translatable mass is medial to the hand wrist or arm. The scaffolding of the frame structure may hold the translatable masses in spaced arrangement to the hand, wrist, or arm such that they flank the hand, wrist, or arm (e.g., located distally to the hand). The axes along which the translatable axes slide may be substantially parallel to each other and substantially perpendicular to the length of the patient's arm. This arrangement of the translatable masses results in the translatable masses to counter-act and dampen extension/flexion motions (e.g., at the wrist) and supination/pronation motions (e.g., at the forearm). When extension or flexion occurs, the first and second translatable masses move in the same direction in response and thereby dampen the tremor motion. When supination or pronation occurs, the first and second translatable masses move in opposite directions, counter-acting the rotational motion created by the supination/pronation.

Additional translatable weight may be included in this design to counteract other vectors of motion, such as flexion/extension motion that creates vertical motion when the arm is held horizontal and outward. For example a third translatable weight having a third axis that is substantially perpendicular to the first and second axes, and is substantially vertical when the arm is held out horizontally may be included in the design to counter-act vertical motion.

As described above, each of the one or more housing members may contain a mass which is translatable along an axis between a proximal limit and a distal limit, and a neutral position midway between the proximal limit and the distal limit, where, following imposition of a force having a component along the axis, a biasing means (i.e., one or more springs) returns the mass to the neutral position. Each housing member of the tremor reduction device of the present invention may include a sliding means upon which the first mass may slidably translate along its axis. The sliding means may include a guide member and/or a bearing member, such as a rail member, magnetic levitation track, or other bearing structure in mechanical connection to the housing. The sliding means may also include a friction reducing material, such as a liquid lubricant (e.g., a hydrocarbon lubricant), a solid lubricant (e.g., graphite), or other appropriate lubricating material.

The frame structure may comprise any material or combination of materials with rigidity sufficient to maintain its shape while transferring force caused by Parkinsonian or essential tremors from the user to the housing members. In some embodiments, the plate may comprise at least one of a rigid polymer or plastic, a metal, a metal alloy, carbon fiber, composite materials, and the like. In some embodiments, the housing member(s) may comprise the same material as the frame structure.

The embodiments of the tremor reduction device of the present invention may include padding material on a surface contacting the skin of the user to prevent abrasion and injury to the wrist. For example, if one or more of the housings for contacts the patients hand, wrist, or forearm, a padding material may be attached to a surface of the housing for positioning between the housing and the skin of the user. The padding material may be memory foam to allow for a snug fit and comfort. In some embodiments, the padding material may be relatively resistant to compression, such that it has a negligible dampening effect on the tremors of the user, such that most or substantially all of the vibrational motion of the tremor is transferred to the housings. For example, the padding may include a material having a Shore A hardness in a range of 20 Shore A to about 60 Shore A (e.g., about 30 Shore A to about 45 Shore A, or any value or range of values therein). Examples of padding materials including vulcanized rubber, polychloroprene rubber, butyl rubber, and other appropriate materials.

Embodiments of the present invention may also include a method of reducing tremors in a limb of a person, including applying or mechanically connecting a tremor reduction device to the limb, as described herein. The tremor reduction device having at least one housing as described herein. Each of the housing members may contain its own mass which is translatable along an axis between a proximal limit and a distal limit set within the housing. A neutral position is defined between the proximal limit and the distal limit. Each of the housings may also contain a biasing means for returning the mass to the neutral position. The mass and biasing means may be sized according to at least one of the horizontal frequency and the vertical frequency of the measured tremors.

Also disclosed herein is a method for reducing tremors in a limb of a person caused by Parkinson's disease or essential tremor utilizing the following steps. First, at least the horizontal and vertical frequency and/or amplitude of the tremors is measured on the person suffering from the condition. Once this information is determined for a particular person suffering from Parkinson's disease or essential tremor, a tremor reduction device is configured. In some embodiments the tremor reduction device has a plurality of housings as described herein (e.g., linked together around the wrist, attached to a frame structure, etc.). Each of the plurality of housing members contains its own mass which is translatable along an axis between a proximal limit and a distal limit set within the housing. A neutral position is defined between the proximal limit and the distal limit. Each of the housings also contains a biasing means for returning the mass to the neutral position. The mass and biasing means are sized according to at least one of the horizontal frequency and the vertical frequency of the measured tremors. The configured tremor reduction device is then placed about the limb of the person.

In further implementations, the frequency and/or amplitude of the tremors are measured along X, Y, and Z axes, and the plurality of housing members are configured to counteract vibrational energy along all three axis by including at least three housing members, each containing translatable masses that move along one of three perpendicular X, Y, Z axes. The arrangement and configuration of the translatable weight axes may be matched to the tremor movement during measurement of the patient's tremors. For example, where the patient was tested during exercises in which the hand is held out horizontally (e.g., in attempting to hold a spoon or other eating utensil), the tremor reduction device may be configured and calibrated such that (1) a first translatable weight will be positioned on an axis that runs medial to lateral with respect to the wrist (assuming anatomical position) and may be calibrated to counteract the vertical tremor motion exhibited by the patient during the test, (2) a second translatable weight will be positioned on an axis that runs anterior to posterior with respect to the wrist (assuming anatomical position) and may be calibrated to counteract the horizontal medial/lateral tremor motion exhibited by the patient during the test, and (3) a third translatable weight will be positioned on an axis that runs proximal to distal with respect to the wrist and may be calibrated to counteract the horizontal anterior/posterior tremor motion exhibited by the patient during the test.

Embodiments of the present invention may be designed to address the tremors suffered by a particular patient. The frequency and amplitude of a person's tremors may be measured on each extremity. Once the frequency and amplitude are known, the size of the mass contained within each of the housing members may be specified. Alternatively, or in addition, the magnitude of the biasing means may be adjusted. For example, if springs are utilized as a biasing means, the spring force may be adjusted by either using springs having a different spring force, or increasing or decreasing the number of springs. The installation of different mass sizes and/or different biasing means is facilitated with embodiments of the present invention which have housings which are adapted to receive a variety of interchangeable weights and springs which may be exchanged within the housing with relative ease.

In some embodiments, the housing members of the present invention may be linked together with straps, belts, netting, or other flexible and light connecting fabric which is suitable for linking the housings and securing the housings about a patient's arm or leg. Embodiments of the invention may further comprise a padding layer between the device itself and patient's arm or leg so that the device fits snugly tight, but is not uncomfortable and does not cause abrasion or injury.

In modeling tremor and the effectiveness of embodiments of the present invention, the inventor herein found that by utilizing the present invention, the magnitude of tremor is substantially reduced. For example, utilizing a machine which models tremor having a frequency of 4 to 5 Hz, the tremor had a horizontal magnitude of 10 millimeters and a vertical magnitude of 21 millimeters without application the tremor reduction device of the present invention. With an embodiment of the invention placed about the “wrist” utilized in the model, the horizontal magnitude was reduced to 3.5 millimeters and the vertical magnitude was reduced to 7.0 millimeters.

Further objects and aspects of the present invention will be apparent from the description provided herein.

### BRIEF DESCRIPTION OF THE DRAWINGS

FIG. 1 shows a front perspective view of an embodiment of the disclosed tremor reduction device placed about the wrist of a user.

FIG. 2 shows a front perspective view of the embodiment depicted in FIG. 1 an embodiment of the invention secured about the wrist of a user with a strap and hook and loop fasteners, with housing covers in place.

FIG. 3 shows a top perspective view of an alternative embodiment.

FIG. 4 shows a front side perspective view of an embodiment utilizing a exterior strap for securing to the wrist of a user.

FIG. 5 shows a side perspective view of the embodiment depicted in FIG. 4.

FIG. 6 shows a front side perspective view of the embodiment depicted in FIG. 4 with the housing covers removed.

FIG. 7 shows a side perspective view of the embodiment depicted in FIG. 4 with the housing covers removed.

FIG. 8 shows a front perspective view of the embodiment depicted in FIG. 4 with the housing covers removed.

FIG. 9 shows a perspective view of a tremor reduction device, according to an embodiment of the present invention.

FIG. 10 shows a perspective view of a tremor reduction device, according to an embodiment of the present invention.

FIG. 11 shows a perspective view of a tremor reduction device, according to an embodiment of the present invention.

FIG. 12 shows a perspective view of a tremor reduction device with the housing covers removed, according to an embodiment of the present invention.

FIG. 13 shows a perspective view of a securing frame, according to an embodiment of the present invention.

### DETAILED DESCRIPTION OF THE DRAWINGS

Reference will now be made in detail to certain embodiments of the invention, examples of which are illustrated in the accompanying drawings. While the invention will be described in reference to these figures and certain implementations and examples of the embodiments, it will be understood that such implementations and examples are not intended to limit the invention. To the contrary, the invention is intended to cover alternatives, modifications, and equivalents that are included within the spirit and scope of the invention as defined by the claims. In the following disclosure, specific details are given to provide a thorough understanding of the invention. References to various features of the “present invention” throughout this document do not mean that all claimed embodiments or methods must include the referenced features. It will be apparent to one skilled in the art that the present invention may be practiced without these specific details or features.

Reference will be made to the exemplary illustrations in the accompanying drawings, and like reference characters may be used to designate like or corresponding parts throughout the several views of the drawings.

The present invention relates to a tremor reduction device. More specifically, the present invention pertains to a wearable band or gauntlet-like frame structure having one or more housings, each enclosing a mass which is translatable along an axis between a proximal limit and a distal limit, where following imposition of a force having a component along the axis, a biasing means (i.e., a spring) returns the mass to a neutral position, e.g., centered on the axis.

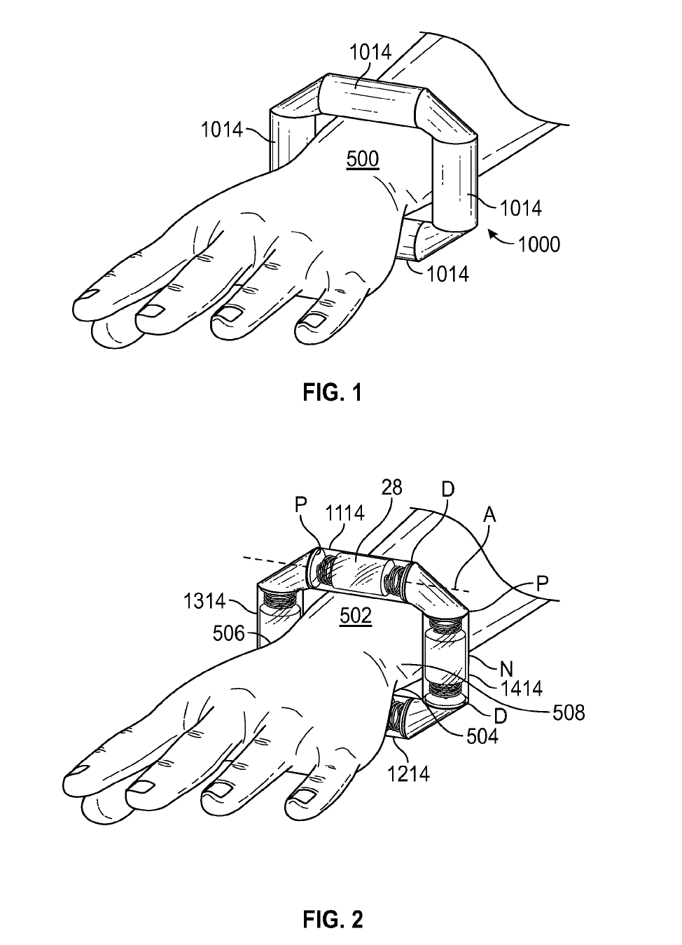

Referring now to the figures, FIG. 1 shows a first embodiment of the disclosed tremor reduction device 1000, as worn around a user's wrist 500. In one embodiment of the invention, the tremor reduction device 1000 has a plurality of housing members 1014 flexibly connected together. The embodiment shown in FIG. 1 has four housing members 1014 flexibly connected together to encircle the user's wrist 500, with a first housing member 1114 adjacent to the top side 502 of the wrist, a second housing member 1214 adjacent to the palm side 504 of the wrist, a third housing member 1314 adjacent the thumb side 506 of the wrist, and a fourth housing member 1414 adjacent to the little finger side 508 of the wrist 500. Each of the housing members 14 contains a mass 28 which is translatable along an axis A between a proximal limit P and a distal limit D, and a neutral position N midway between the proximal limit P and the distal limit D, wherein, following imposition of a force having a component along the axis, a biasing means returns the mass to the neutral position.

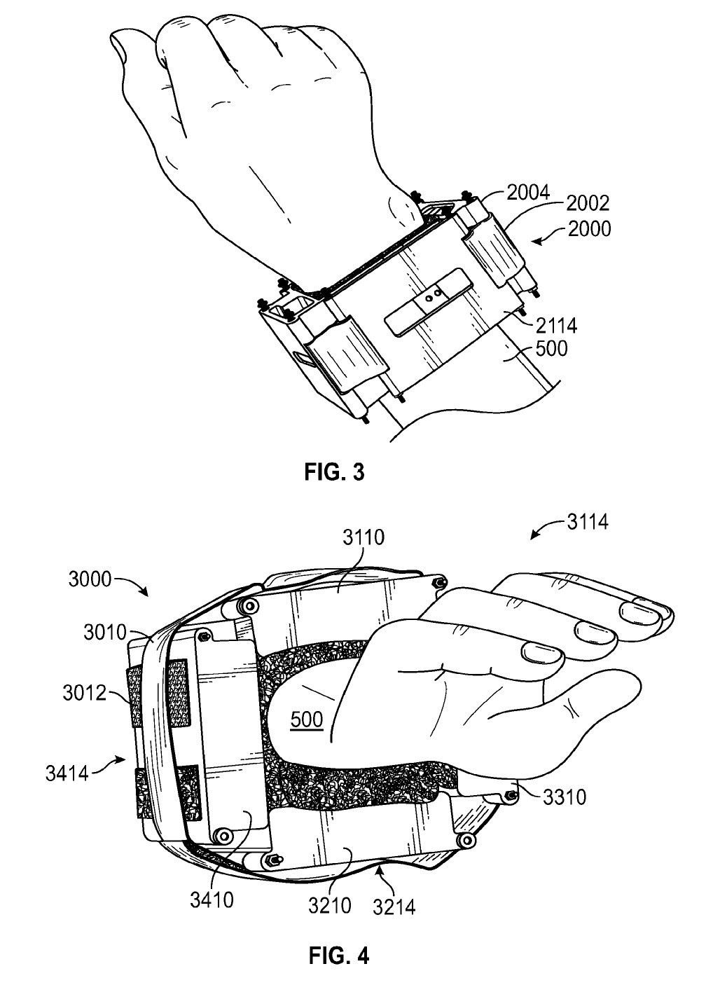

A second embodiment of the tremor reduction device is depicted in FIG. 3. The second embodiment 2000 is secured about the user's wrist 500 by a strap 2002 which loops around strap posts of adjacent housing members 2114. Strap 2002 will have an attachment mechanism, such as a buckle, clasp, snap buttons, or hook and loop fasteners, for securing the strap and enclosing the device around the user's wrist. Housing members 2114 of the second embodiment 2000 are identical to the housing members 3114 of the third embodiment 3000 and are described in detail below.

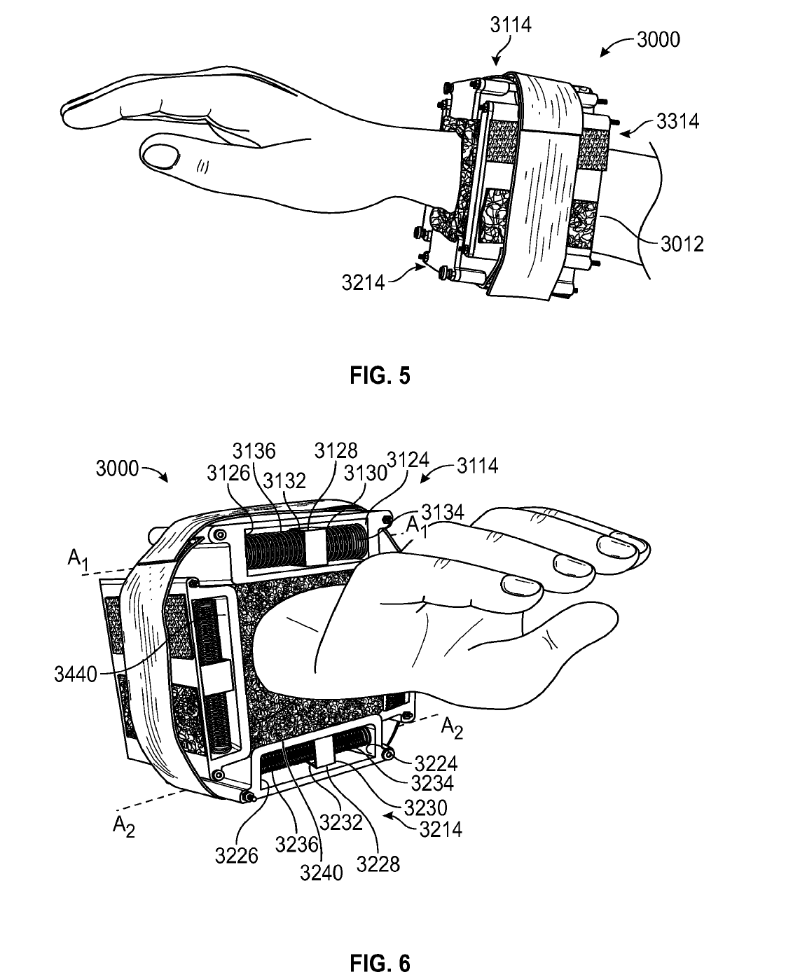

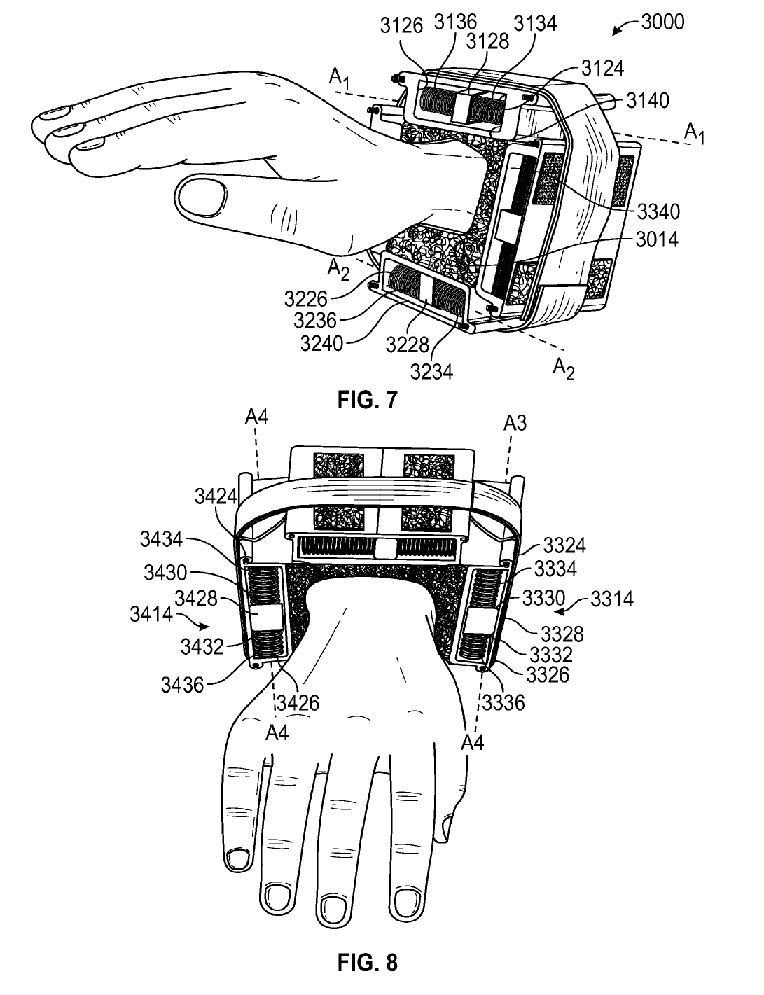

FIGS. 4-8 depict a third embodiment 3000 of the tremor reduction device wherein first housing member 3114 has a housing cover 3110, second housing member 3214 has a housing cover 3210, third housing member 3314 has a housing cover 3310, and fourth housing member 3414 has a housing cover 3410. Third embodiment 3000 may be secured around the user's wrist 500 by a continuous strap 3010 which wraps around the outsides of the housing members 3114, 3214, 3314 and 3414. Each housing cover 3110, 3214, 3314 and 3414 may have attachment means, such as piece of hook and loop fastener, 3012, for further securing continuous strap 3010. The inside surface of the tremor reduction device may comprise a layer of memory foam 3014 which may be disposed between any embodiment 1000, 2000, 3000 of the tremor reduction device and the wrist 500 of the user.

The housing members of the tremor reduction device 3000 may have the following configuration. First housing member 3114 may have a first proximal sidewall 3124 and a first distal sidewall 3126, with the first proximal sidewall 3124 and the first distal sidewall 3126 in opposite facing spaced-apart relation. A first mass 3128 is disposed between the first proximal side wall 3124 and the first distal side wall 3126. The first mass 3128 has a first proximal side 3130 and a first distal side 3132. A first proximal spring 3134 is disposed between the first proximal side wall 3124 and the first proximal side 3130 of the first mass 3128. A first distal spring 3136 is disposed between the first distal side wall 3126 and the first distal side 3132 of the first mass 3128. First mass 3128 is translatable along axis A.sub.1 between a first proximal limit set by first proximal side wall 3124 and first distal side 3132, where following imposition of a first force having a component along axis A.sub.1, first proximal spring 3134 and first distal spring 3136 return first mass 3128 to a first neutral position at an approximate midpoint between first proximal side wall 3124 and first distal side wall 3126. FIGS. 6-8 depict first mass 3128 at the first neutral position. It is to be appreciated that during a tremor episode the first mass 3128 may be in near constant oscillation between the first proximal side wall 3124 and the first distal side wall 3126, such that the first mass 3128 does not come to a static condition at the first neutral position. The first neutral position is achieved when no external forces—such as a tremor—act upon the first mass.

First proximal sidewall 3124 and first distal sidewall 3126 may be attached to or may be integral to a first base plate 3140. First base plate 3140 may have a sliding means upon which the first mass 3128 may slidably translate. The sliding means may include a rail member, magnetic levitation, air suspension or a layer of a friction reducing medium, such as balls.

Likewise, second housing member 3214 may have a second proximal sidewall 3224 and a second distal sidewall 3226, with the second proximal sidewall 3224 and the second distal sidewall 3226 in opposite facing spaced-apart relation. A second mass 3228 is disposed between the second proximal side wall 3224 and the second distal side wall 3226. The second mass 3228 has a second proximal side 3230 and a second distal side 3232. A second proximal spring 3234 is disposed between the second proximal side wall 3224 and the second proximal side 3230 of the second mass 3228. A second distal spring 3236 is disposed between the second distal side wall 3226 and the second distal side 3232 of the second mass 3228. Second mass 3228 is translatable along axis A.sub.2 between a second proximal limit set by second proximal side wall 3224 and second distal side 3232, where following imposition of a second force having a component along axis A.sub.2, second proximal spring 3234 and second distal spring 3236 return second mass 3228 to a second neutral position at an approximate midpoint between second proximal side wall 3224 and second distal side wall 3226. FIGS. 6-8 depict second mass 3228 at the second neutral position. It is to be appreciated that during a tremor episode the second mass 3228 may be in near constant oscillation between the second proximal side wall 3224 and the second distal side wall 3226, such that the second mass 3228 does not come to a static condition at the second neutral position. The second neutral position is achieved when no external forces—such as a tremor—act upon the second mass.

Second proximal sidewall 3224 and second distal sidewall 3226 may be attached to or may be integral to a second base plate 3240. Second base plate 3240 may have a sliding means upon which the second mass 3228 may slidably translate. The sliding means may include a rail member, magnetic levitation, air suspension or a layer of a friction reducing medium, such as balls.

Likewise, third housing member 3314 may have a third proximal sidewall 3324 and a third distal sidewall 3326, with the third proximal sidewall 3324 and the third distal sidewall 3326 in opposite facing spaced-apart relation. A third mass 3328 is disposed between the third proximal side wall 3324 and the third distal side wall 3326. The third mass 3328 has a third proximal side 3330 and a third distal side 3332. A third proximal spring 3334 is disposed between the third proximal side wall 3324 and the third proximal side 3330 of the third mass 3328. A third distal spring 3336 is disposed between the third distal side wall 3326 and the third distal side 3332 of the third mass 3328. Third mass 3328 is translatable along axis A.sub.3 between a third proximal limit set by third proximal side wall 3324 and third distal side 3332, where following imposition of a third force having a component along axis A.sub.3, third proximal spring 3334 and third distal spring 3336 return third mass 3328 to a third neutral position at an approximate midpoint between third proximal side wall 3324 and third distal side wall 3326. FIGS. 6-8 depict third mass 3328 at the approximate third neutral position. It is to be appreciated that during a tremor episode the third mass 3328 may be in near constant oscillation between the third proximal side wall 3324 and the third distal side wall 3326, such that the third mass 3328 does not come to a static condition at the third neutral position. The third neutral position is achieved when no external forces—such as a tremor—act upon the third mass.

Third proximal sidewall 3324 and third distal sidewall 3326 may be attached to or may be integral to a third base plate 3340. Third base plate 3340 may have a sliding means upon which the third mass 3328 may slidably translate. The sliding means may include a rail member, magnetic levitation, air suspension or a layer of a friction reducing medium, such as balls.

Likewise, fourth housing member 3414 may have a fourth proximal sidewall 3424 and a fourth distal sidewall 3426, with the fourth proximal sidewall 3424 and the fourth distal sidewall 3426 in opposite facing spaced-apart relation. A fourth mass 3428 is disposed between the fourth proximal side wall 3424 and the fourth distal side wall 3426. The fourth mass 3428 has a fourth proximal side 3430 and a fourth distal side 3432. A fourth proximal spring 3434 is disposed between the fourth proximal side wall 3424 and the fourth proximal side 3430 of the fourth mass 3428. A fourth distal spring 3436 is disposed between the fourth distal side wall 3426 and the fourth distal side 3432 of the fourth mass 3428. Fourth mass 3428 is translatable along axis A.sub.4 between a fourth proximal limit set by fourth proximal side wall 3424 and fourth distal side 3432, where following imposition of a fourth force having a component along axis A.sub.4, fourth proximal spring 3434 and fourth distal spring 3436 return fourth mass 3428 to a fourth neutral position at an approximate midpoint between fourth proximal side wall 3424 and fourth distal side wall 3426. FIGS. 6-8 depict fourth mass 428 at the fourth neutral position. It is to be appreciated that during a tremor episode the fourth mass 3428 may be in near constant oscillation between the fourth proximal side wall 3424 and the fourth distal side wall 3426, such that the fourth mass 3428 does not come to a static condition at the fourth neutral position. The fourth neutral position is achieved when no external forces—such as a tremor—act upon the fourth mass.

Fourth proximal sidewall 3424 and fourth distal sidewall 3426 may be attached to or may be integral to a fourth base plate 3440. Fourth base plate 3440 may have a sliding means upon which the fourth mass 3428 may slidably translate. The sliding means may include a rail member, magnetic levitation, air suspension or a layer of a friction reducing medium, such as balls.

As shown in the Figures, the axes of translation of each side of the device are in parallel alignment. That is, axis A.sub.1 is in parallel alignment with axis A.sub.2 and axis A.sub.3 is in parallel alignment with axis A.sub.4. In this configuration, the translation of the first mass 3128 along axis A.sub.1 and the translation of the second mass 3228 along axis A.sub.2 collectively reduce tremor along a first translational direction which is parallel to axis A.sub.1 and axis A.sub.2. Likewise, the translation of the third mass 3328 along the axis A.sub.3 and the translation of the fourth mass 3428 along axis A.sub.4 reduce tremor along a second translational direction which is parallel to axis A.sub.3 and A.sub.4. the sum of the forces generated by the translation of first mass 3128 along axis A.sub.1, second mass 3228 along axis A.sub.2, third mass 3328 along axis A.sub.3 and fourth mass along axis A.sub.4 reduce tremor having a rotational direction.

The different embodiments 1000, 2000, 3000 may have interchangeable weight for use as the required mass (e.g., 3128, 3218, 3318, and 3418 for the third embodiment) for providing the required force for neutralizing the force generated by the tremor. Likewise, the spring size and stiffness can be adjusted as necessary to provide the required biasing force for translation of each of the masses. Embodiments of the device may have features which facilitate interchanging different weights and springs to allow empty housings to be configured as required for the needs of a particular patient.

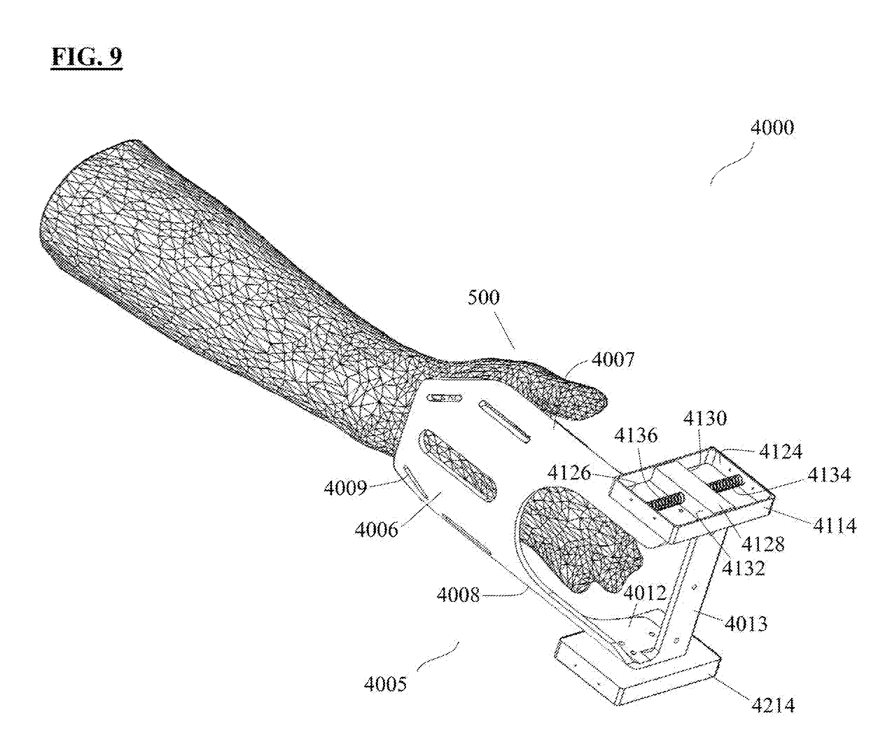

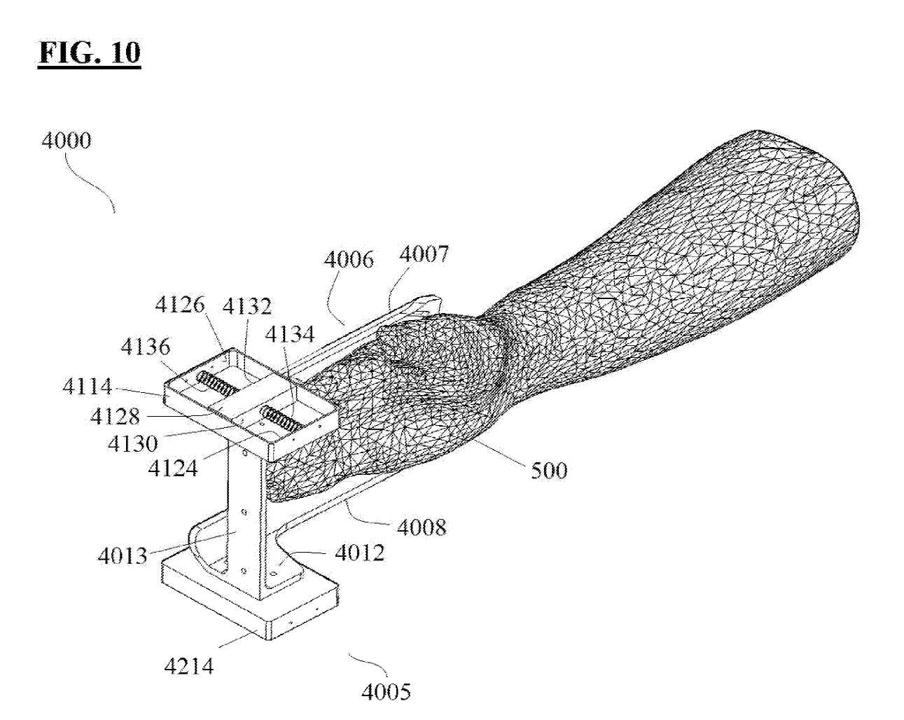

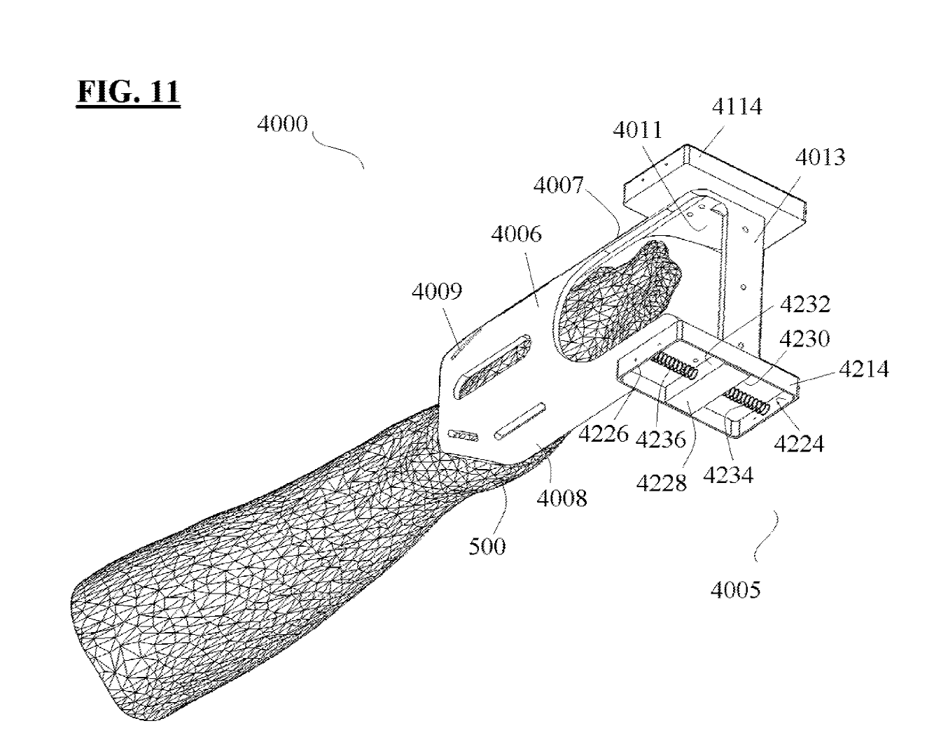

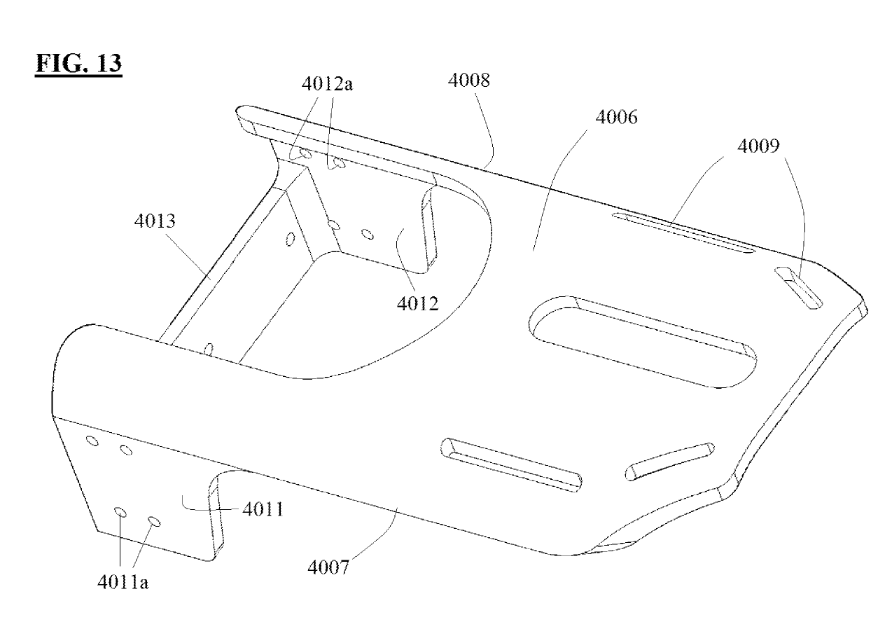

As seen in FIGS. 9-13, some embodiments of the present invention may provide a tremor reduction device 4000 comprising a frame structure 4005 and a plurality of housing members 4114, 4214. The frame structure 4005 may comprise a coupling plate 4006 and a plurality of brackets 4011, 4012 for mounting the plurality of housing members 4114, 4214. The frame structure 4005 may comprise a rigid material (e.g., plastic, metal, composite, etc.) operable to maintain its shape while transferring force caused by tremors from the user to the plurality of housing member 4114, 4214.

The coupling plate 4006 may comprise a substantially flat shape with first curving edge 4007 and a second curving edge 4008, the first and second curving edges 4007, 4008 curving around the sides of a user's hand 500. The plate 4006 may further comprise an attachment element 4009 for securing straps to the plate 4006, the straps being operable to hold the frame structure 4005 in place on the user's hand 500. The attachment element 4009 may comprise a plurality of slots for the straps to pass through.

The frame structure 4005 may include a scaffolding 4010 that extends distally from the coupling plate 4006 and includes attachment brackets 4011 and 4012. The scaffolding 4010 may extend from the first curving edge 4007 and the second curving edge 4008, such that the first bracket 4011 and second bracket 4012 are positioned at or near the distal end of the user's hand. The scaffolding may include a reinforcing crossbar 4013 between the first and second brackets 4011 and 4012. The first bracket 4011 may comprise a substantially flat shape having a first plurality of holes 4011a and the second bracket may comprise a substantially flat shape having a second plurality of holes 4012a. The first plurality of holes 4011a may be operable to receive a first securing device (not shown) for securing the first housing member 4114 to the first bracket 4011, and the second plurality of holes 4012a may be operable to receive a second securing device (not shown) for securing the second housing member 4214 to the second bracket 4012.

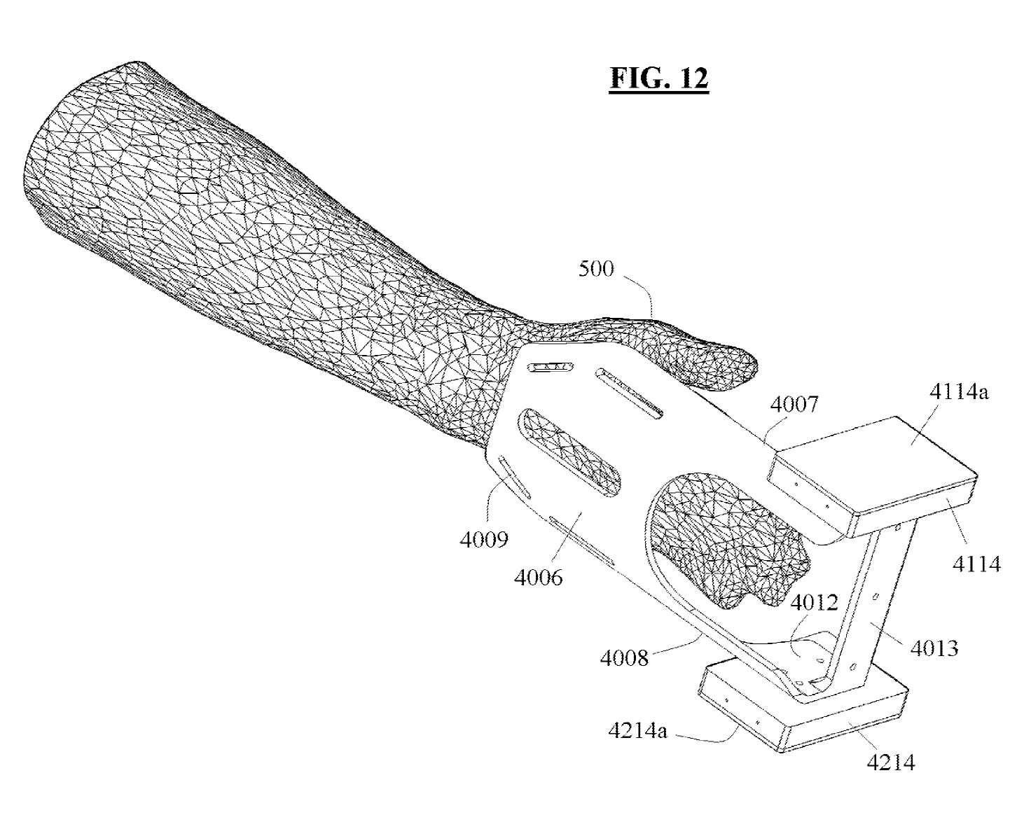

The first housing member 4114 may enclose a first sidewall 4124 and a second sidewall 4126, where the first sidewall 4124 and the second sidewall 4126 are in opposite facing, spaced-apart relation. A first mass 4128 may be disposed between the first proximal side wall 4124 and the first distal side wall 4126, wherein the first mass 4128 may have a first side 4130 and a second side 4132. A first spring 4134 may be disposed between the first side wall 4124 and the first side 4130 of the first mass 4128, and a second spring 4136 may be disposed between the second side wall 4126 and the second side 4132 of the first mass 4128. The first sidewall 4124 and the second sidewall 4126 may be attached to or may be integral to a first base plate. The first base plate may have a sliding means 4135 upon which the first mass 4128 may slidably translate. The sliding means 4135 may include a rail member, a track, magnetic levitation track, or other structure along which the first mass 4128 may slide. The sliding means may also include a layer of solid or liquid lubricating material. The first housing member 4114 may comprise a removable first housing cover 4114a. The first housing cover 4114a is depicted in FIG. 12, but is removed in FIGS. 9 through 11 for the purpose of clarity.

The second housing member 4214 may have a first sidewall 4224 and a second sidewall 4226 in opposite facing, spaced-apart relation. A second mass 4228 may be disposed between the first side wall 4224 and the second side wall 4226, wherein the second mass 4228 has a first side 4230 and a second side 4232. A first spring 4234 may be disposed between the first side wall 4224 and the first side 4230 of the second mass 4228, and a second spring 4236 may be disposed between the second side wall 4226 and the second side 4232 of the second mass 4228. The first sidewall 4224 and second sidewall 4226 may be attached to or may be integral to a second base plate. The second base plate may have a sliding means 4235 upon which the second mass 4228 may slidably translate. The sliding means may include a rail member, a track, magnetic levitation track, or other structure along which the second mass 4228 may slide. The sliding means may also include a layer of solid or liquid lubricating material. The second housing member 4214 may comprise a removable second housing cover 4214a. The second housing cover 4214a is depicted in FIG. 12, but is removed in FIGS. 9 through 11 for the purpose of clarity.

The first mass 4128 and the second mass 4228 may each move along an axis when a tremor applies a horizontal force to the securing frame, the first mass 4128 moving along a first axis which is substantially parallel to a longitudinal (horizontal in the embodiment shown) axis of the first housing member 4114, and the second mass moving along a second axis which is substantially parallel to a longitudinal (horizontal in the embodiment shown) axis of the second housing member 4214. In the embodiment shown in FIGS. 9 through 12, the first spring 4134 and the second spring 4136 may be mounted within the first housing 4114 so as to be co-located with the first axis, and the first spring 4234 and second spring 4236 may be mounted within the second housing 4214 so as to be co-located with the second axis.

In some embodiments, the first axis and the second axis may be in parallel with each other, and linear forces generated by the first mass 4128 and the second mass 4228 may significantly reduce the effect of a tremor which has a force parallel with the first axis and the second axis (i.e., a side-to-side motion of the user's hand 500). In such embodiments, the sum of a rotational force generated by the first mass 4128 and second mass 4228 may also reduce the effect of a tremor which has a rotational direction (i.e., a twist of the user's hand 500).

Also disclosed herein is a method for reducing tremors in a limb of a person caused by Parkinson's disease or essential tremor utilizing the following steps. First the horizontal and vertical frequency of the tremors is measured on the person suffering from the condition. Once this information is determined for a particular person suffering from Parkinson's disease or essential tremor, a tremor reduction device is configured, such as one of the embodiments 1000, 2000, 3000 of the presently disclosed device. As described above, the tremor reduction device has a plurality of housings linked together in a closed loop configuration. Each of the plurality of housings contains its own mass which is translatable along an axis between a proximal limit and a distal limit set within the housing. A neutral position is defined between the proximal limit and the distal limit. Each of the housings also contains a biasing means for returning the mass to the neutral position. The mass and biasing means are sized according to the horizontal frequency and the vertical frequency of the measure tremors. The configured tremor reduction device is then placed about the limb of the person.

It is to be understood that variations, modifications, and permutations of embodiments of the present invention, and uses thereof, may be made without departing from the scope of the invention. It is also to be understood that the present invention is not limited by the specific embodiments, descriptions, or illustrations or combinations of either components or steps disclosed herein. The embodiments were chosen and described in order to best explain the principles of the invention and its practical application, to thereby enable others skilled in the art to best utilize the invention and various embodiments with various modifications as are suited to the particular use contemplated. Although reference has been made to the accompanying figures, it is to be appreciated that these figures are exemplary and are not meant to limit the scope of the invention. It is intended that the scope of the invention be defined by the claims appended hereto and their equivalents.

1. A tremor reduction device worn by a user, the device comprising:
a. a frame structure for attaching said tremor reduction device to a portion of said user's body;
b. at least one housing member attached to said frame structure;
c. a mass contained in said at least one housing member, said mass being translatable along an axis between a proximal limit and a distal limit; and
d. a biasing mechanism operable to return said mass to a neutral position between the proximal limit and the distal limit when said mass is displaced from said neutral position by a force having a component along the axis.

2. The tremor reduction device of claim 1, wherein the at least one housing member comprises a first housing member enclosing a first mass, and a second housing member enclosing a second mass.

3. The tremor reduction device of claim 2, further comprises a third housing member enclosing a third mass.

4. The tremor reduction device of claim 2, wherein the device is operable to reduce a horizontal component of a Parkinsonian or essential tremor.

5. The tremor reduction device of claim 2, wherein the device is operable to reduce a rotational component of a Parkinsonian or essential tremor.

6. The tremor reduction device of claim 3, wherein the device is operable to reduce a vertical component of a Parkinsonian or essential tremor.

7. The tremor reduction device of claim 2, wherein the first housing member comprises a first sliding means and the first mass slidably translates along the first sliding means.

8. The tremor reduction device of claim 2, wherein each of the first and second housing members each comprise a sliding means and the masses slidably translate along the sliding means.

9. The tremor reduction device of claim 7, wherein the first sliding means comprises a first rail member.

10. The tremor reduction device of claim 2, wherein the first mass translates along a first axis, and the second mass translates along a second axis.

11. The tremor reduction device of claim 3, wherein the third mass translates along a third axis.

12. The tremor reduction device of claim 10, wherein the first axis is in parallel alignment with the second axis.

13. A tremor reduction device worn by a user, the device comprising:
a. a first housing member comprising a first mass translatable along a first axis between a first proximal limit and a first distal limit, the first mass having a first neutral position midway between the first proximal limit and the first distal limit, when said first mass is displaced from said first neutral position by a force having a component along the first axis, a first biasing means returns the first mass to the first neutral position; and
b. a second housing member comprising a second mass translatable along a second axis between a second proximal limit and a second distal limit, the second mass having a second neutral position midway between the second proximal limit and the second distal limit, when said second mass is displaced from said neutral position by a force having a component along the second axis, a second biasing means returns the second mass to the second neutral position;
c. a frame structure operable to be attached to a portion of the user's body, the first and second housing members being attached to said frame structure.

14. The tremor reduction device of claim 14, wherein said frame structure is attached to a portion of the arm of the user, and includes a scaffolding positioned to which the first and second housings are attached, said scaffolding being at a spaced distance from the user's arm.

15. The tremor reduction device of claim 14, wherein the first mass translates along a first axis, and the second mass translates along a second axis, and said first and second axes are parallel.

16. (canceled)

17. The tremor reduction device of claim 15, further comprises a third housing member enclosing a third mass, wherein said third mass translates along a third axis.

18. The tremor reduction device of claim 15, wherein said third axis is perpendicular to said first axis and said second axis.

19. The tremor reduction device of claim 15, wherein said frame structure includes a coupling plate operable to contact and attach to the portion of the user's arm, said coupling plate having a contoured surface for complementary fitting to the portion of the user's arm.

20. A tremor reduction device worn by a patient, the device comprising:
a. an attachment structure operable to be attached to a portion of an arm of said patient, the attachment structure having a scaffolding;
b. a first translatable weight in mechanical connection to the scaffolding, said first translatable weight operable to move along a first axis and to dampen component vibrations of a Parkinsonian or essential tremor along said first axis; and
c. a second translatable weight in mechanical connection to the scaffolding, said second translatable weight operable to move along a second axis and to dampen component vibrations resulting from said Parkinsonian or essential tremor along said second axis.

21. The tremor reduction device of claim 20, wherein said scaffolding positions said first and second translatable weights at a pre-determined distance from said arm of said patient.

22. The tremor reduction device of claim 20, wherein said first axis and said second axis are substantially parallel.

23. (canceled)

24. (canceled)

25. (canceled)

26. The tremor reduction device of claim 20, wherein the first translatable weight is located laterally to the arm and the second translatable weight is located medially to the arm of the patient, such that the first translatable weight and the second translatable weight flank the arm.

27. The tremor reduction device of claim 26, wherein said first and second axes are parallel to each other and perpendicular to the length of the arm.

28. The tremor reduction device of claim 27, wherein said first and second weights move in the same direction during flexion and extension movements of the wrist.

29. The tremor reduction device of claim 27, wherein said first and second translatable weights move in opposite directions during supination and pronation movements of the wrist.
